(19) United States
(12) Patent Application Publication
(10) Pub. No.: US 2018/0272134 A1
(43) Pub. Date: Sep. 27, 2018

(54) Microstimulator Having Body-Mounted Electrodes and Remote Electrode Leads

(71) Applicant: Boston Scientific Neuromodulation Corporation, Valencia, CA (US)

(72) Inventors: Samuel Tahmasian, Glendale, CA (US); Matthew Lee McDonald, Pasadena, CA (US); William Morgan, Stevenson Ranch, CA (US); Rafael Carbunaru, Valley Village, CA (US); Jillian Doubek, Los Angeles, CA (US)

(21) Appl. No.: 15/914,758

(22) Filed: Mar. 7, 2018

Related U.S. Application Data
(60) Provisional application No. 62/474,488, filed on Mar. 21, 2017.

(51) Int. Cl. A61N 1/36; A61N 1/372; A61N 1/375

(52) U.S. Cl. CPC A61N 1/36125; A61N 1/0529; A61N 1/3754; A61N 1/37211

(57) ABSTRACT

An implantable pulse generator (IPG) is disclosed herein. The IPG includes two or more body-mounted electrodes that can be independently programmed to provide stimulation at the location of implantation. The IPG also includes connectors for connecting one or more leads configured with electrode arrays for providing stimulation remote from the IPG. The IPG can be implanted at one location in a patient's body where stimulation is to be delivered and the one or more remote leads can be implanted in additional locations. The disclosed IPG with both body-mounted and remote electrodes reduces the charging complexity of having two microstimulators implanted. The remote lead(s) may be either permanently attached to the IPG or may be removeably attached.

### CROSS REFERENCE TO RELATED APPLICATIONS

This is a non-provisional application based on U.S. Provisional Patent Application Ser. No. 62/474,488, filed Mar. 21, 2017, which is incorporated by reference in its entirety, and to which priority is claimed.

### FIELD OF THE INVENTION

The present invention relates to a rigid support structure for an implantable medical device.

### INTRODUCTION

Implantable stimulation devices are devices that generate and deliver electrical stimuli to body nerves and tissues for the therapy of various biological disorders. Examples include pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. FIG. 1A shows an implantable stimulation device as may be used for spinal cord stimulation or deep brain stimulation. Such a device typically includes an Implantable Pulse Generator (IPG) 10, which includes a hermetically sealed case 12 formed of a conductive material such as titanium and a header portion 28, which is typically a biocompatible polymer or a ceramic material. The case 12 typically holds the circuitry and battery 14 (FIG. 1C) necessary for the IPG 10 to function. Some IPGs can be powered via external RF energy and without a battery. The IPG 10 is coupled to one or more arrays 18 of electrodes (E1-E16). The array(s) 18 of electrodes are disposed on leads 22. The leads 22 house the individual signal wires 24 coupled to each electrode. In the illustrated embodiment, there are eight electrodes on each lead 22, although the number of leads and electrodes is application specific and therefore can vary. The leads bodies 22 are coupled to a lead connector 26 within the header portion 28 of the IPG 10 via cables 20. The header typically includes electrical feed throughs that provide a conduction path between the lead connector 26 and the hermetically sealed case.

As shown in the cross-section of FIG. 1C, the IPG 10 typically includes a printed circuit board (PCB) 30, along with various electronic components 32 mounted to the PCB 30, some of which are discussed subsequently. Two coils (more generally, antennas) are show in the IPG 10: a telemetry coil 34 used to transmit/receive data to/from an external controller (not shown); and a charging coil 36 for charging or recharging the IPG's battery 14 using an external charger. Charging and data coils and supporting electronic components for operating an IPG are described in U.S. Pat. Nos. 6,516,227, and 8,738,138 issued Feb. 4, 2003 and May 27, 2014, respectively and U.S. Publication No. 2015/0157861A1, published Jun. 11, 2015.

An external charger (not shown) is typically used to wirelessly convey power to the IPG 10, which power can be used to recharge the IPG's battery 14. The transfer of power from the external charger is enabled by a primary charging coil in the charger. The external charger may also include user interface, including touchable buttons and perhaps a display and a speaker, allows a patient or clinician to operate the external charger.

FIG. 2 shows a first embodiment 201 of implantable stimulation device implanted in a patient for deep brain stimulation and a second embodiment 202 implanted in the patient for spinal cord stimulation. Deep brain stimulation may be indicated to treat a variety of neurological symptoms, such as tremor, stiffness, rigidity and slowed movement associated with Parkinson's disease or essential tremor. For deep brain stimulation, the IPG 10 is typically embedded in the in the patient's chest inferior to the clavicle. The signal wires 24 are routed beneath the skin of the patient's neck and head and the leads 18 are implanted into the patient's brain 32.

Spinal cord stimulation may be used to treat chronic back pain. For spinal cord stimulation, the IPG 10 is typically embedded in the in the patient's buttock and the leads 18 are implanted into the patient's spinal column. IPGs may also be used in other therapies, such as sacral nerve stimulation to treat various modalities of incontinence and occipital nerve stimulation for treating migraine headaches.

A problem with implantable stimulation devices utilizing IPGs, such as those illustrated in FIGS. 1 and 2, is that the IPG is quite large, having a volume of about 20 cm³ or more, for example. The IP's size limits the number of places on a patient's body that it can be easily implanted. Another problem is that the metallic case 12 can complicate certain diagnostic imaging techniques, such as magnetic resonance imaging (MRI). Thus, a smaller IPG having less metallic material would be beneficial.

### BRIEF DESCRIPTION OF THE DRAWINGS

FIGS. 1A-1C show different views of an implantable pulse generator, a type of implantable medical device (IMD), in accordance with the prior art.

FIG. 2 shows IMDs used for deep brain stimulation and for spinal cord stimulation.

FIGS. 3A-3D show a micro implantable pulse generator (mIPG).

FIGS. 4A and 4B show cross section views of an electronics compartment of an mIPG.

FIGS. 5A-5E show a molded shell for an mIPG and an mIPG contained within such a molded shell.

FIGS. 6A and 6B show an embodiment of an mIPG having a connector stack and a lead attachable to the mIPG via the connector stack.

FIGS. 7A-7C show an embodiment of an mIPG having body electrodes.

FIGS. 8A and 8B show embodiments of a molded shell having embedded conductors for an mIPG.

FIG. 9 shows a molded shell having embedded conductors for an mIPG.

FIGS. 10A and 10B show an mIPG having multiple electrode types.

FIG. 11 shows an mIPG having antennas embedded in a molded shell.

### DESCRIPTION

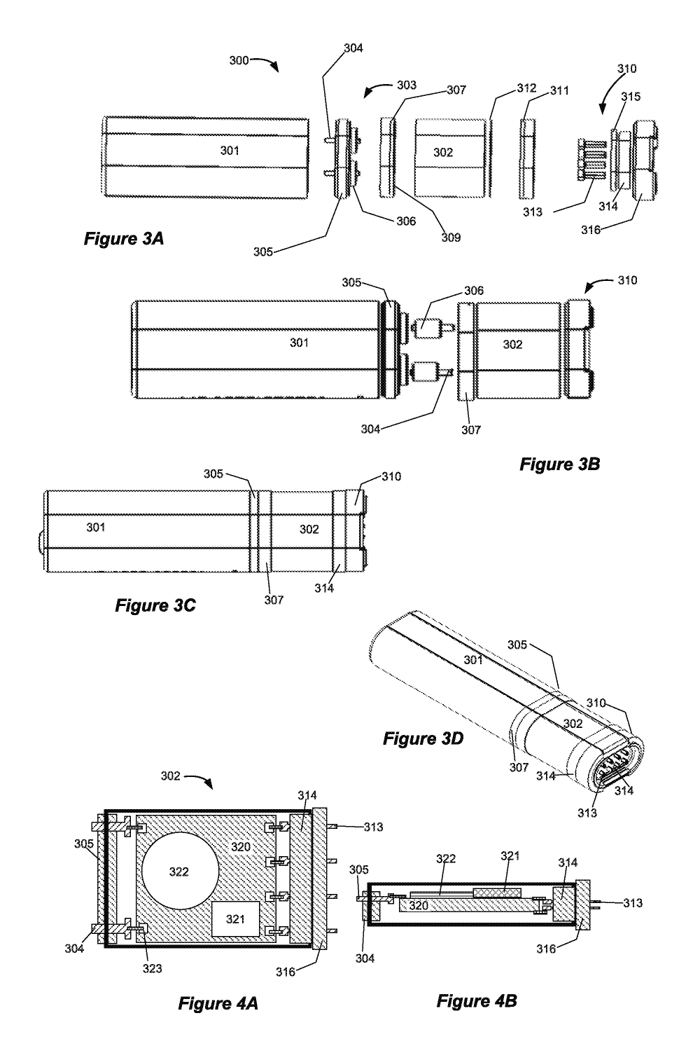

FIGS. 3A-D show an embodiment of an implantable pulse generator, referred to herein as a micro implantable pulse generator (mIPG) 300. The illustrated mIPG includes a battery case 301 and an electronics compartment 302. The battery case 301 is typically made of a medical grade metal material, such as titanium, a titanium alloy, or stainless steel and is configured to contain a power supply, such as a battery for powering the mIPG. Some embodiments of the mIPG shown in FIG. 3A-D differ from than the prior art IPGs discussed above in the sense that the battery and the supporting electronics are contained within separate compartments. Thus, the mIPG 300 is modular. According to some embodiments, the battery case 301 contains no electronic components other than a battery and conductors that provide a conductive path to the electronics compartment 302.

The battery may be a rechargeable battery or may be a primary battery (i.e., a battery that is not rechargeable). Examples of suitable batteries include batteries based on metal hydride or lithium ion technology. Suitable batteries and methods for charging them (if applicable) are described in U.S. Pat. Nos. 6,516,227, and 8,738,138 issued Feb. 4, 2003 and May 27, 2014, respectively and U.S. Publication No. 2015/0157861A1, published Jun. 11, 2015, referenced above. Each of those documents are incorporated herein by reference for the purpose of describing IPG electronics, power supply, charging, and telemetry.

The electronics compartment 302 can be made of a biocompatible non-metallic material such as a ceramic material. The electronics compartment 302 may be configured to enclose the coil(s) and electronic components that are necessary for operating the mIPG 300. According to other embodiments, one or more coils may be disposed external to the electronics compartment 302, as described below.

The battery case 301 and the electronics compartment 302 are joined by a battery feedthrough assembly 303. The battery feedthrough assembly 303 can comprise conducting battery pins 304, which extend through a battery cover 305 and into the electronics compartment 302. The battery pins 304 can be electrically insulated from the battery cover 305 by insulators 306, which are made of an insulating material such as glass or ceramic. The connection between the battery cover 305 and the electronics compartment 302 can include a brazing connector 308 and braze ring 309 for laser welding the two components together.

The electronics compartment 302 connects to an electrode feedthrough assembly 310 for connecting to various therapeutic electrodes, which are discussed below. The electronics compartment 302 may be laser welded to the electrode feedthrough assembly 310 via a braze connector 311 and a braze ring 312. The electrode feedthrough assembly may include one or more mIPG pin electrodes 313, which extend through an insulator 314. The insulator 314 may be a ceramic or glass material, for example. The feedthrough may be supported and held in place with one or more flanges, such as a thin metallic flange 315 and a feedthrough flange 316. Such flanges may also be used to attach electrode assemblies to the electrode feedthrough assembly 310.

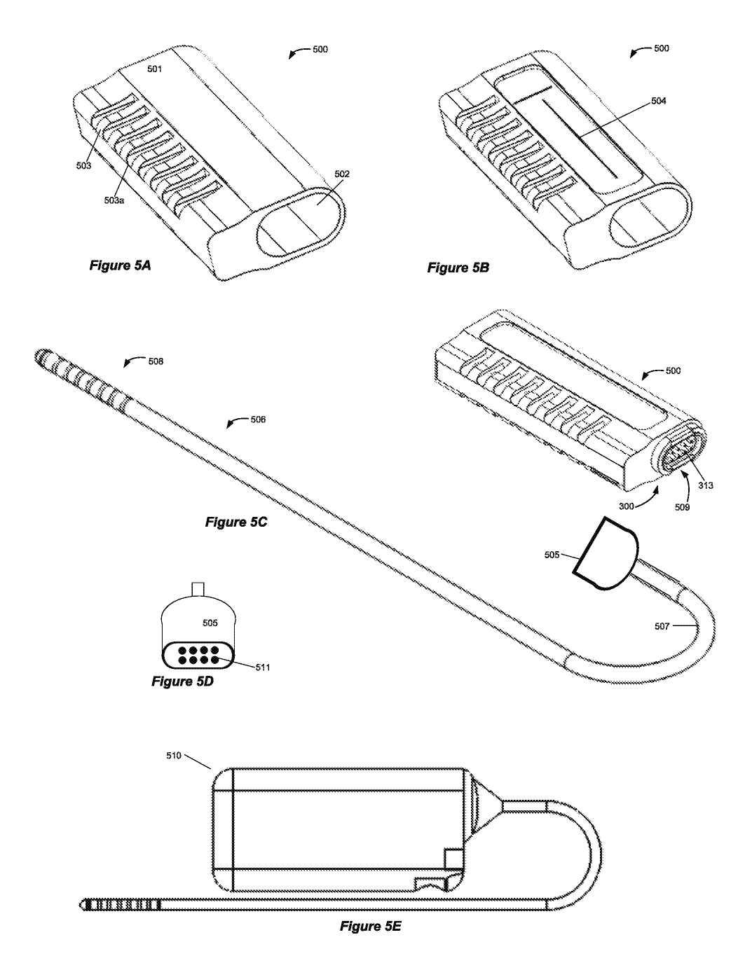

FIGS. 4A and 4B show plan and lateral cross sections, respectively, of the electronics compartment 302. The electronics compartment 302 can contain a printed circuit board (PCB) 320, upon which electronic components 321 may be mounted. The electronic components 321 may include pulse generation circuitry mounted in the form of microprocessors, integrated circuits, capacitors, and other electronic components. The electronics compartment may also comprise one or more charging/telemetry coils 322 and associated charging/telemetry circuitry. Again, the electronics are not discussed in detail here; the reader is referred to in U.S. Pat. Nos. 6,516,227, and 8,738,138 issued Feb. 4, 2003 and May 27, 2014, respectively and U.S. Publication No. 2015/0157861A1, published Jun. 11, 2015, referenced above. The battery pins 304 and the mIPG pin electrodes 313 may be electrically connected to bond pads 323 on the PCB 320. The particular electronic components 321 and one or more coils 322 are described in the patent/application documents referenced above.

According to some embodiments, the mIPG 300 may have a volume of less than 10 cm³, less than 5 cm³, or less than 3 cm³. According to some embodiments, the mIPG 300 has a total volume on the order of about 3 cm³. For example, the length (L) may be about 2 cm, the width (W) about 1.5 cm, and the height (H) about 1 cm. These dimensions are only an example and are not limiting. The point is that embodiments of the mIPG can be much smaller than the IPGs discussed in the Introduction section, above.

FIGS. 5A-5E show a molded shell 500 configured to contain the mIPG 300. The molded shell 500 comprises a body 501 that is typically made from a rigid biocompatible polymeric material such as polyurethane or high density polyethylene (HDPE), or the like. The molded shell 500 provides structural rigidity between the electronics compartment 302 and the other components of the mIPG and protects. In the mIPG illustrated in FIG. 5, the other component is the battery case 301. As explained in more detail below, the molded shell 500 may contain components instead of, or in addition to, a battery case. In general, the molded shell 500 provides a rigid support, i.e., it is a rigid shell, that holds the modular components of the mIPG together.

As mentioned above, the battery case 301 and the electronics compartment 302 can be laser welded together. But the molded shell 500 substantially increases the structural stability of the combination. In other words, the battery case 301 and electronics compartment 302 are less likely to flex or bend with respect to each other when they are at least partially contained within the molded shell 500. According to some embodiments, the molded shell 500 contains essentially 100% of the volume of the mIPG's modular components. According to other embodiments, the molded shell 500 may contain less than 100% of the volume of the mIPG's modular components, for example 70%, 60%, 50%, 40%, 30%, 20% or 10%.

The molded shell 500 includes an opening 502 to provide access to the mIPG pin electrodes 313. According to some embodiments, the molded shell may include ridges 503 to facilitate suturing the mIPG into the patient's tissue, as explained below in more detail.

According to some embodiments, the body 501 of the molded shell 500 includes an opening 504 to provide access to one or more electrodes, such as a case electrode. IPGs utilizing a case electrode are known in the art. See, e.g., U.S. Pat. No. 6,516,227. In embodiments wherein the electronics compartment 302 is made of a non-conducting material such as a ceramic, the battery case 301 may serve as a case electrode. Alternatively, one or more conductors may be attached to the body of the mIPG and exposed via the opening 504, as explained in more detail below. In such an embodiment, the electrodes may be referred to as body electrodes.

FIG. 5C shows the molded shell 500 with the mIPG 300 contained inside it. The mIPG 300 is configured within the molded shell 500 so that the mIPG pin electrodes 313 form an mIPG connector 509 (illustrated as a male connector), which can connect with a connector 505 (illustrated as a female connector) for connecting a lead 506 to the mIPG. It should be noted that the illustrated embodiment features eight pin electrodes 313. However, any number of pin electrodes may be present, for example, four, sixteen, or thirty-two pin electrodes.

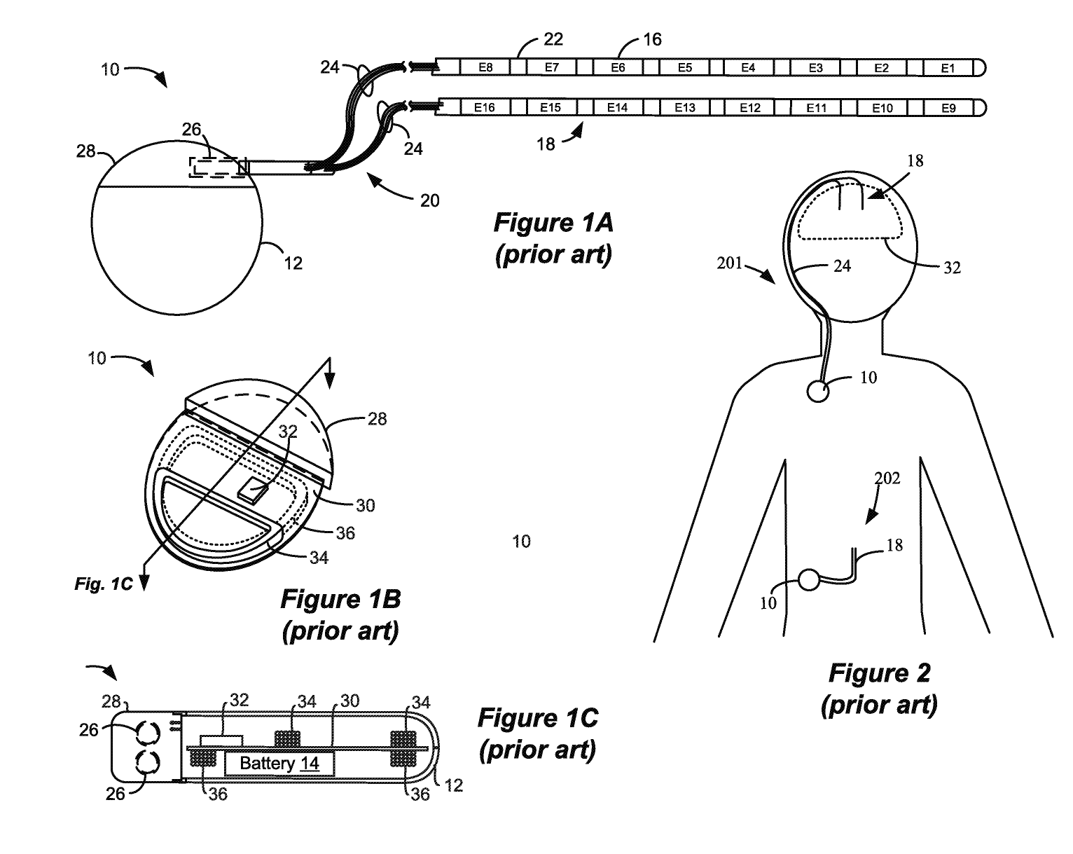

FIG. 5D illustrates another view of the connector 505, which comprises female receptacles 511, which are configured to mate with the mIPG pin electrodes 313. The connector 505 attaches to a cable 507, which attaches to the lead 506. The lead 506 is similar to the lead 18 of the prior art device discussed in the background section above (see FIG. 1A). The lead 506 supports an array of electrodes 508.

Once the connector 505 is connected to the mIPG 300, the entire assembly can be over-molded within a soft coating 510, as shown in FIG. 5D. Examples of suitable over-molding materials include soft, biocompatible polymeric materials, such as silicone. The soft coating 510 acts as another barrier for protection against potential leakage of non-biocompatible material. The soft coating 510 may include an opening (not shown) to provide access to a case electrode or other body electrode(s) if the mIPG includes such electrode(s). The soft coating 510 also holds the connector 505 in place. When the mIPG assembly is sutured into a patient's tissue, the soft coating 510 material can deform into the gaps 503a between the ridges 503 of the molded shell 500. Thus, the coated mIPG assembly can be sutured in place without needing to make suture holes in either the molded shell 500 or the soft coating material.

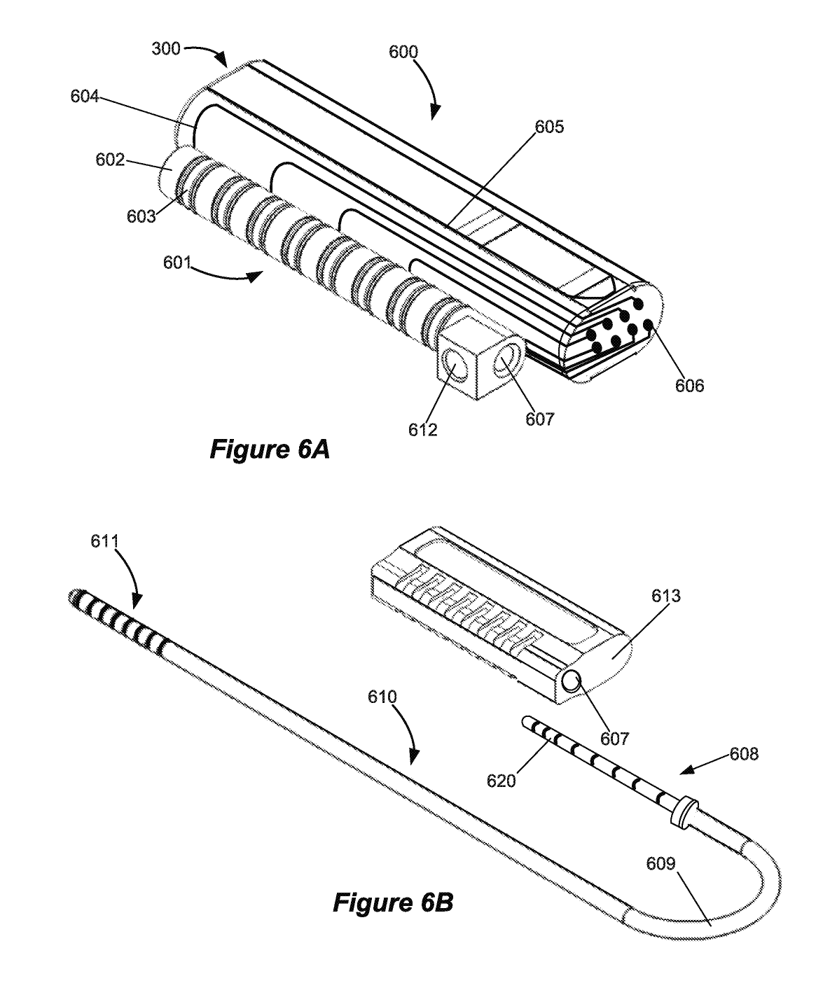

FIGS. 6A and 6B show components of another embodiment of an mIPG assembly 600. The mIPG assembly 600 includes an mIPG 300 and a connector stack 601 for attaching a lead 610 to the mIPG. The connector stack 601 contains a plurality of conducting housings 602, each of which contain a connector spring contact. Each housing 602 is separated by a non-conducting seal 603 and makes electrical contact with a conducting trace 604 supported upon a flexible electrode assembly 605. The flexible electrode assembly 605 may be made of a polymer, for example. The conducting traces 604 may be applied to the flexible electrode assembly by sputtering, for example. The conducting traces 604 connect to contacts 606 on the flexible electrode assembly 605. The contacts 606 are configured to contact the mIPG pin electrodes 313 (FIG. 3D).

The connector stack 601 includes an opening 607 for receiving a connector 608 that is attached to the lead 610 via a cable 609. The lead 610 supports an array of electrodes 611. When the connector 608 is inserted into the opening 607, contact patches 620 on the connector 608 contact corresponding connector spring contacts within the connector stack 601, which, in turn, are in electrical contact with corresponding mIPG pin electrodes 313 via the intervening housings 602 and conducting traces 604.

The connector stack 601 also includes an opening 612 configured to receive a set screw (not show) for holding the connector 608 in place once it is connected. Thus, the connector 608 is removable from the connector stack 601 upon loosening the set screw. The mIPG assembly 600 can be contained within a rigid molded shell 613, similar to the molded shell 500 shown in FIGS. 5A-5C (common features are not renumbered here). The molded shell 613 can then be over-molded in a soft material, such as silicone (not shown).

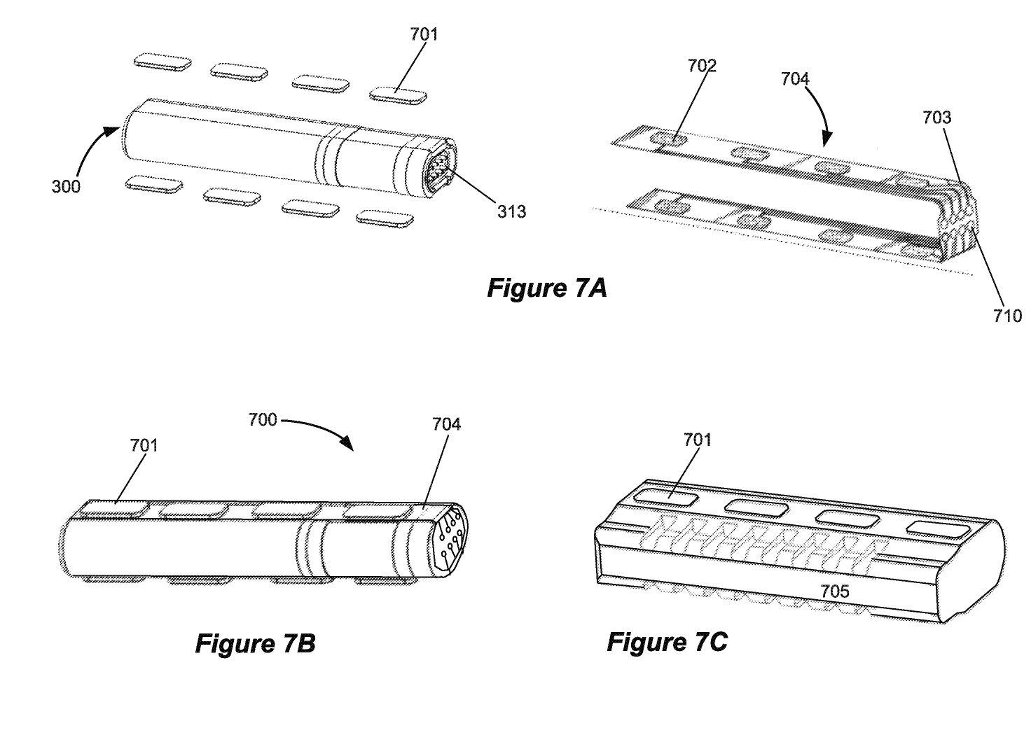

FIGS. 7A-7C illustrate another embodiment on an mIPG assembly 700 wherein an mIPG 300 is configured with a plurality of body electrodes 701. Note that the mIPG assembly 700 is different from the embodiments illustrated in FIGS. 5 and 6 in that the mIPG assembly 700 does not include a connector for attaching to a cable/lead. Instead, the body electrodes 701 provide the therapeutic currents. As used herein, the term “body electrodes” refers to stimulation electrodes that are configured upon the body of the mIPG and that provide stimulation in the location where the mIPG is implanted. Thus, the mIPG assembly 700 is intended to be implanted at the location within the patient's body where therapy is to be delivered. This is in contrast to stimulation electrodes that are configured upon a lead (such as 506 of FIG. 5 and 610 of FIG. 6) attached to the mIPG by a cable and are configured to deliver stimulation remotely from the mIPG. Such electrodes may be referred to herein as “remote electrodes.”

The pulse generation circuitry of the mIPG may control various parameters of the stimulation current applied to the body electrodes 701; for example, it may control the frequency, pulse width, amplitude, burst patter, duty cycle, etc., applied to the stimulation site. Various of the body electrodes 701 may be selected as cathodes or as anodes. The embodiment of an mIPG assembly 700 illustrated in FIGS. 7A-7C has eight body electrodes 701. It will be appreciated that each of the electrodes 701 can operate independently, i.e., they can be independently programed to provide various therapeutic current patterns. For example, one or more of the electrodes 701 may act as a current source and others of the electrodes 701 may act as a current sink. Moreover, one or more of the body electrodes 701 may be shorted together to form a larger electrode or a case electrode.

The body electrodes 701 are placed in contact with a flexible electrode assembly 704, upon which is deposited conducting patches 702, conducting traces 703, and contacts 710. The contacts 710 are configured to align with the mIPG pin electrodes 313 when the mIPG and flexible electrode assembly are combined, thereby providing an electrical path between the mIPG pin electrodes 313 and the body electrodes 701. Alternatively, the body electrodes may be deposited directly upon the flexible electrode assembly in lieu of the conducting patches 702.

FIG. 7B illustrates how the mIPG 300, the body electrodes 701, and the flexible electrode assembly 704 fit together. FIG. 7C shows the mIPG assembly encased within a molded shell 705. Note that the molded shell 705 includes openings to allow access to the body electrodes 701. The mIPG/molded shell assembly can be over-molded within a soft coating material, such as silicone (not shown). Openings to allow access to the body electrodes 701 may be included in any over-molded coating.

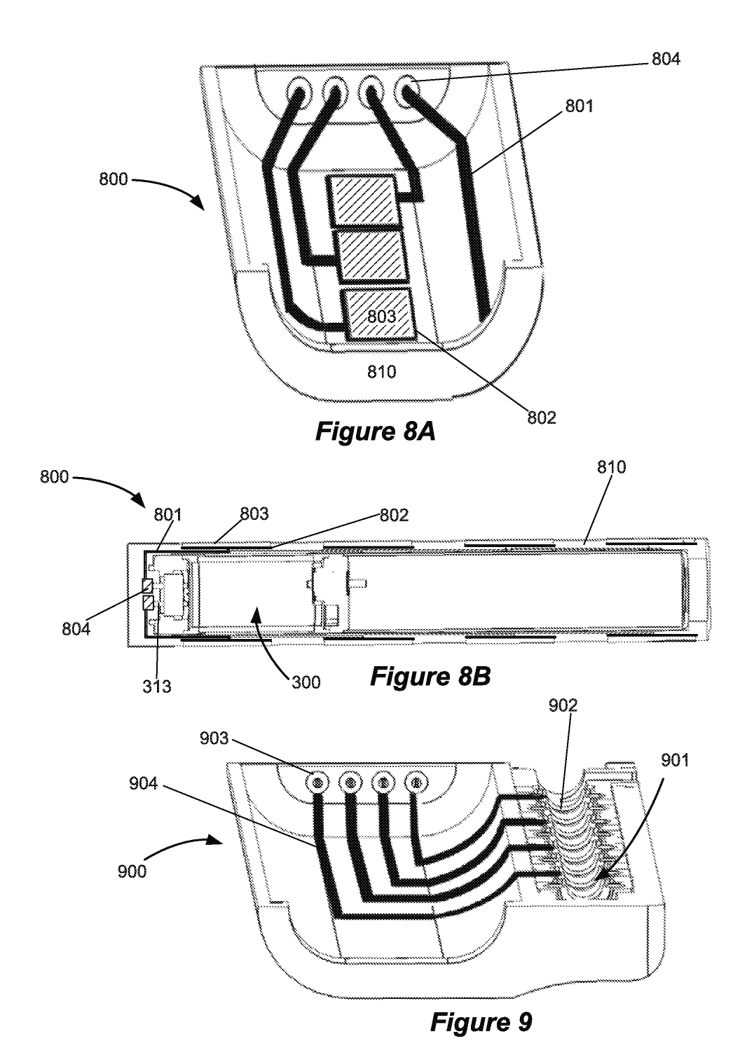

FIGS. 8A and 8B show an alternative embodiment of a molded shell 800. FIG. 8A shows a cross section of the molded shell 800 in perspective view and FIG. 8B shows a cross section lateral view of the molded shell with relevant portions of an mIPG 300 included for reference. The molded shell 800 has conducting traces 801 and conducting patches 802 embedded into the body 810 of the molded shell. The conducting patches 802 are positioned around openings 803, which are configured to provide access to body electrodes when an mIPG assembly is contained within the molded shell. The conducting traces 801 are also connected to contacts 804, which are positioned to connect with the mIPG pin electrodes 313 when an mIPG is contained within the molded shell 800. Essentially, the embedded conducting traces 801, conducting patches 802, and contacts 804 eliminate the need to use a flexible electrode assembly 704, as illustrated in FIGS. 7A and 7B, to maintain electrical contact between the mIPG pin electrodes 313 and body electrodes.

FIG. 9 shows a cross section of an embodiment of a molded shell 900 with a compartment 901 configured to contain a connector stack, such as connector stack 601 of FIG. 6A. Conducting patches 902 are embedded within the compartment 901 for making electrical contact with the spring housings 602 of the connector stack. The conducting patches 902 are electrically connected to contacts 903 via conducting traces 904 embedded in the molded shell 900. The contacts 903 are positioned to make electrical contact with the mIPG pin electrodes 313 of an mIPG. As with the molded shell 800 illustrated in FIGS. 8A and 8B, the molded shell 900 essentially eliminates the need to use a flexible electrode assembly to contact an mIPG. Embodiments utilizing molded shells having conducting patches and traces embedded therein, such as illustrated in FIGS. 8 and 9, greatly simplify the construction of mIPG assemblies.

mIPG assemblies having three different electrode configurations have been described above. Namely, those electrode configurations are (1) a lead permanently attached directly to the mIPG pin electrodes, as illustrated in FIGS. 5C and 5D, (2) a lead removably attached to a connector stack, as illustrated in FIGS. 6A and 6B, and (3) body electrodes, as illustrated in FIGS. 7A through 7C. Moreover, the configurations implementing a connector stack or body electrodes may be implemented either using flexible electrode assemblies (i.e., 604 of FIG. 6A or 704 of FIG. 7A) or they may be implemented using a molded shell having conducting patches and conducting traces embedded therein, as illustrated in FIGS. 8 and 9.

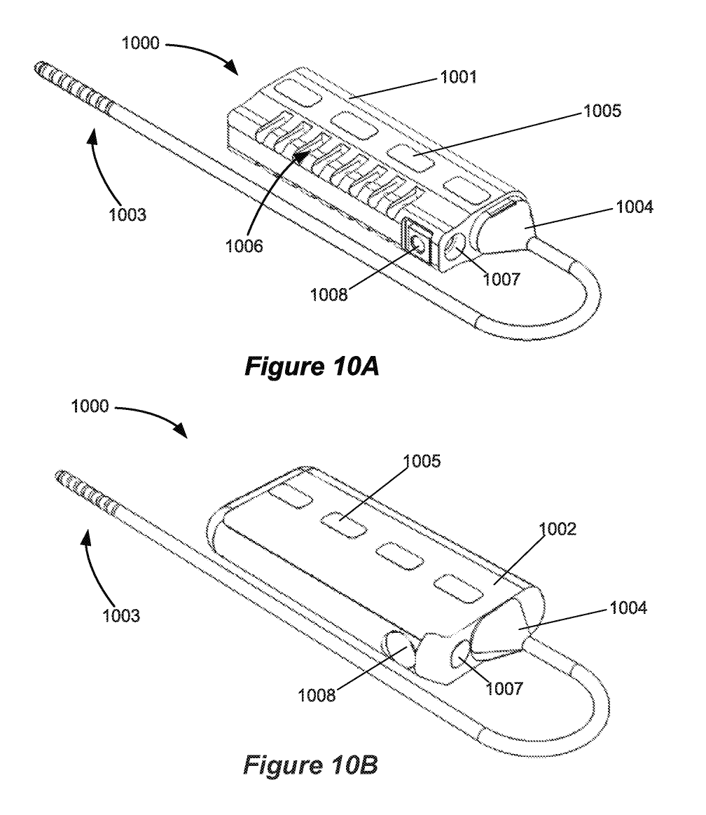

FIGS. 10A and 10B illustrate an mIPG assembly 1000 having all three electrode configurations. FIG. 10A illustrates the mIPG assembly 1000 contained within a rigid molded shell 1001, while FIG. 10B illustrates the mIPG assembly/molded shell assembly overcoated with a soft coating material 1002, such as silicone. The mIPG assembly 1000 includes a permanently attached lead 1003 attached to the mIPG assembly 1000 via a connector 1004. Mating pins within connector 1004 may attach to one or more of the mIPG pin electrodes (313 of FIG. 3D) of the mIPG.

The mIPG assembly 1000 can also include one or more body electrodes 1005. Electrical contact between the body electrodes 1005 and the mIPG pin electrodes (313 of FIG. 3D) of the mIPG may be provided either by a flexible electrode assembly (704 of FIG. 7A) or by conducting patches and conducting traces (803 and 801 of FIG. 8A, respectively). In embodiments having both a permanently attached lead 1003 and body electrodes 1005, mating pins within the connector 1004 of the lead may attach to some of the mIPG pin electrodes (313 of FIG. 3D) of the mIPG and the contacts for the body electrodes (710 of FIG. 7A, for example) may attach to other of the mIPG pin electrodes. In other words, some of the mIPG pin electrodes may be dedicated to operating the permanently attached lead 1003 and others of the mIPG electrodes may be dedicated to operating the body electrodes 1005. According to other embodiments, particular individual mIPG pin electrodes 313 can connect both to mating pins within the connector 1004 and contacts for the body electrode.

The mIPG assembly 1000 can also include a connector stack 1006 (contained within the molded shell 1001). The molded shell 1001 includes an opening 1007 so that a connector (e.g., 620 of FIG. 6B) for a lead can connect with the connector stack 1006. The molded shell 1001 may also include another opening 1008 so that the connector can be secured in place with a set screw, as explained above. As with the body electrodes, the connector stack 1006 may be connected to the mIPG pin electrodes either by a flexible electrode assembly or by conducting pads and traces embedded within the molded shell 1001.

In sum, the mIPG assembly 1000 may contain any combination of electrode types: a permanently attached lead, body electrode(s), and/or a connector stack-connected lead. Each of the types of electrodes can be independently programmed with respect to each other. The ability to have multiple types of electrodes connected to a single mIPG provides significant therapeutic flexibility. For example, a physician may treat debilitating headaches in a patient using occipital nerve stimulation (ONS), during which stimulation of multiple nerves may be indicated. In such a case, the physician may implant the mIPG near one nerve or nerve center so that body electrodes can provide stimulation to that location and implant an attached lead near another nerve or nerve center. Other use cases include combined spinal cord stimulation (SCS) and peripheral nerve stimulation (PNS). Using a single mIPG to stimulate both locations simplifies the process because there is only a single battery to charge and mIPG to program.

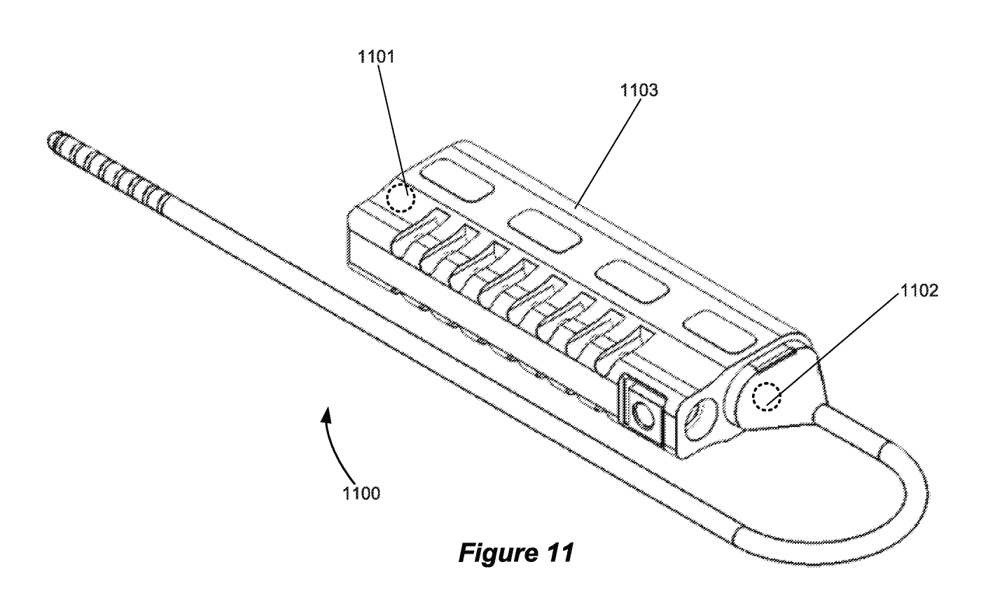

FIG. 11 illustrates a further embodiment of an mIPG assembly 1100, wherein one or more antennas, 1101 and 1102, are embedded in the molded shell 1103. The antennas 1102 and/or 1103 may be embedded in a similar manner as described with respect to the electrical contacts and electrical traces illustrated in FIGS. 8 and 9. FIG. 11 illustrates only two possible locations for the antennas 1102 and/or 1103; they can generally be embedded anywhere within the molded shell 1103. The antennas 1102 and/or 1103 may be coils, for example either charging coils or telemetry coils, as is known in the art. According to other embodiments, the antennas 1102 and/or 1103 may be radio antennas, for example, Bluetooth antennas or the like.

It should be noted that the mIPG embodiments illustrated above include a battery compartment for housing a primary or rechargeable battery. However, alternative embodiments may not include a battery and may instead receive power from an external power source that couples transcutaneously to one or more coils within the mIPG assembly. Such external powering is described, for example, in U.S. Pat. No. 8,155,752, which is incorporated herein by reference for the disclosure of transcutaneous coupling between an external power source and a coil within an implantable device. Thus, antennas 1102 and/or 1103 may be power coils for coupling to an external power source for powering the mIPG.

Generally, the modular devices and methodologies described herein allow components that would traditionally be enclosed within a hermetically sealed casing to be moved outside of that casing and structurally supported using a rigid shell structure. Thus, the size of the casing can be reduced.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover equivalents that may fall within the spirit and scope of the present invention as defined by the claims.

What is claimed is:

1. An implantable pulse generator (IPG) comprising:
a device body;
a plurality of body electrodes disposed upon the device body; and
one or more connectors for attaching one or more remote electrodes to the device body.

2. The IPG of claim 1, wherein the device body comprises a battery compartment connected to an electronics housing.

3. The IPG of claim 2, wherein the battery compartment comprises a metallic material.

4. The IPG of claim 2, wherein the electronics housing comprises a glass or ceramic material.

5. The IPG of claim 1, wherein the IPG has a volume of less than 3 cm³.

6. The IPG of claim 2, further comprising a shell comprising a polymeric material at least partially enclosing both the battery compartment and the electronics housing and having openings for the body electrodes.

7. The IPG of claim 6, wherein the body electrodes electrically contact electronic components inside the electronics housing via a flexible electrode assembly, the flexible electrode assembly comprising:
a flexible substrate supporting a conducting path comprising conducting patches connected to conducting traces connected to contacts, each disposed upon a flexible substrate, wherein
the body electrodes are disposed upon the conducting patches, and wherein
the contacts are configured to contact pin electrodes connected electronic components inside the electronics housing.

8. The IPG of claim 6, wherein the body electrodes electrically contact electronic components inside the electronics housing via conductors embedded in the shell, wherein the conductors terminate with contacts configured to contact pin electrodes connected to electronic components inside the electronics housing.

9. The IPG of claim 2, wherein the connector for attaching one or more remote electrodes comprises pin electrodes connected to electronic components inside the electronics housing, wherein the pin electrodes are configured to mate with a remote electrode assembly.

10. The IPG of claim 9, further comprising an overmolded coating disposed upon the IPG, the overmolded coating securing the connector for attaching one or more remote electrodes in contact with the remote electrode assembly.

11. The IPG of claim 2, wherein the connector for attaching one or more remote electrodes comprises a connector stack configured to connect with a remote electrode assembly.

12. The IPG of claim 11, wherein the connector stack comprises a plurality of conducting housings, each conducting housing containing a canted coil spring and wherein the conducting housings electrically contact electronic components inside the electronics housing via a flexible electrode assembly, the flexible electrode assembly comprising:
a flexible substrate supporting a conducting path comprising conducting patches connected to conducting traces connected to contacts, each disposed upon a flexible substrate, wherein
the conducting housings contact the conducting patches, and wherein the contacts are configured to contact pin electrodes connected electronic components inside the electronics housing.

13. The IPG of claim 11, further comprising a shell comprising a polymeric material at least partially enclosing the battery compartment, the electronics housing, and the connector stack.

14. The IPG of claim 13, further comprising an overmolded coating disposed upon the shell, wherein the shell and the overmolded coating both comprise an opening for the connector stack, configured so that a connector of a remote electrode assembly can be plugged into and unplugged from the connector stack.

15. A medical device comprising:
an implantable pulse generator (IPG) comprising:
a device body,
a plurality of body electrodes disposed upon the device body, and
one or more connectors for attaching one or more remote electrode leads to the IPG; and
and at least one remote electrode lead connected to the IPG.

16. The medical device of claim 15, wherein the remote electrode lead is permanently connected to the IPG.

17. The medical device of claim 16, wherein the remote electrode lead is secured to the IPG by overcoating the IPG and a portion of the remote electrode lead in a coating material.

18. The medical device of claim 15, wherein the remote electrode lead is removeably connected to the IPG.

19. The medical device of claim 15, wherein parameters of each of the plurality of body electrodes are independently programmable.

20. The medical device of claim 15, wherein the plurality of body electrodes can be programmed to act together as a single electrode.
